(19) United States
(12) Patent Application Publication
(10) Pub. No.: US 2022/0134301 A1
(43) Pub. Date: May 5, 2022

(54) PH-SENSITIVE CAPSULE AND RELEASE SYSTEM

(71) Applicant: Ohio State Innovation Foundation, Columbus, OH (US)

(72) Inventors: Chao LI, Columbus, OH (US); Xiaolei GUO, Columbus, OH (US); Gerald FRANKEL, Columbus, OH (US)

(21) Appl. No.: 17/434,211

(22) PCT Filed: Mar. 12, 2020
(86) PCT No.: PCT/US2020/022413

§ 371 Date: Aug. 26, 2021

Related U.S. Application Data
(60) Provisional application No. 62/817,315, filed on Mar. 12, 2019.

(51) Int. Cl. B01J 13/10; A61K 9/51; C23C 22/40; B01J 19/26

(52) U.S. Cl. CPC B01J 13/10; A61K 9/51; C23F 11/04; B01J 19/26; C23C 22/40

(57) ABSTRACT

A pH-sensitive release system comprising a capsule capable of releasing an agent in both low pH environments and high pH environments. The capsule encapsulates an agent and comprises at least two weak polyelectrolytes (e.g., PEI and PAA). The capsule responds to both low and high pH changes in the local environment by releasing the agent. The agent may include a corrosion inhibitor and may help prevent or ameliorate the effects of corrosion.

### CROSS-REFERENCE TO RELATED APPLICATIONS

This application is a national stage application of International Application No. PCT/US2020022413, filed on Mar. 12, 2020 entitled PH-SENSITIVE CAPSULE AND RELEASE SYSTEM, which claims the benefit of U.S. Provisional Application No. 62/817,315, filed on Mar. 12, 2019 entitled PH-SENSITIVE CAPSULE AND RELEASE SYSTEM, the contents of which are incorporated herein by reference in their entireties.

### TECHNICAL FIELD

The innovation relates to a pH sensitive release system, e.g., a corrosion inhibitor release system capable of releasing inhibitors in both low pH environments and high pH environments,

### BACKGROUND

Some smart coatings used for corrosion protection can respond to stimuli resulting from the corrosion process and release functional species inside the coatings to repair damage and/or inhibit further corrosion. Redox reactions during the corrosion process can result in a change in the pH within a coating. The pH change that occurs during corrosion has been used as the stimulus to trigger the release of these functional species. However, current coatings are limited such that they release agents under either acidic or basic pH. Thus, any corrosion protection occurs only in the area where there is either a net anodic or cathodic reaction, leading to a limited corrosion protection performance.

### SUMMARY

The following presents a simplified summary of the innovation in order to provide a basic understanding of some aspects of the innovation. This summary is not an extensive overview of the innovation. It is not intended to identify key/critical elements of the innovation or to delineate the scope of the innovation. its sole purpose is to present some concepts of the innovation in a simplified form as a prelude to the more detailed description that is presented later.

According to an aspect, the innovation provides a pH-sensitive release system capable of releasing an agent in both low pH and high pH environments. In one embodiment, the pH-sensitive release system is a corrosion inhibitor release system capable of releasing inhibitors in both low pH environments and high pH environments. This corrosion inhibitor release system is able to heal voids/defects created by inhibitor consumption, thus, improving the long-term corrosion performance of coatings. It is also easier and less expensive to manufacture.

In one embodiment, the coating may comprise an encapsulated corrosion inhibitor. In one embodiment, the corrosion inhibitor system may include a micro-container or a nano-container comprising two weak polyelectrolytes. In one embodiment, the polyelectrolytes may be polyethylenimine (PEI) and polyacrylic acid (PAA).

In one aspect, the innovation provides a method of forming a corrosion inhibitor release system comprising forming a micro-container or a nano-container that encapsulates a corrosion inhibitor.

### BRIEF DESCRIPTION OF THE DRAWINGS

FIG. 1 is a schematic diagram of an embodiment of a method according to the innovation of the fabrication of vanadate-loaded nano-/micro-capsules using electrospray technique.

FIG. 2 is a schematic diagram of an embodiment of a method according to the innovation of the fabrication of a corrosion protection system using electrospray technique.

FIG. 3 is a diagram depicting testing of PEI/PAA complexes of varying molar ratios in varying pH environments.

FIG. 4 depicts results of testing of PEI/PAA complexes having a molar ratio of 1:1.

FIG. 5 depicts results of testing of PEI/PAA complexes having a molar ratio of 2:1.

FIG. 6 depicts results of testing of PEI/PAA complexes having a molar ratio of 1:2.

### DETAILED DESCRIPTION

In the following description, for purposes of explanation, numerous specific details are set forth in order to provide a thorough understanding of the subject innovation. It may be evident, however, that the innovation can be practiced without these specific details.

According to an aspect, the innovation provides a pH-sensitive release system. The pH-sensitive release system comprises a capsules (e.g., PEI/PAA capsules) that respond to both low and high pH changes in the local environment. The pH in the local environment decreases to acidic values in regions where anodic reactions are localized owing to hydrolysis of metal cations. In contrast, the pH increases to alkaline values in regions where cathodic reactions occur. In some cases, the cathodic reactions occur in aqueous environments.

In embodiments, the capsules of the pH-sensitive release system according to the innovation may be useful in most any environment in which a pH change is indicative of a condition that would be improved by release of an encapsulated agent. For example, an increase or decrease of pH in an environment may be indicative of a risk for damage caused by corrosion. Release of a corrosion inhibitor in either of the circumstances (e.g., a change to low or high pH) could help to mitigate or prevent corrosion damage. In another example, a biological condition that manifests with an increase or decrease in pH could be treated by the release of a medication/compound to treat the condition with the use of a capsule according to the innovation. In yet another example, the capsule according to the innovation may be used in agricultural contexts. For, example, the capsules may encapsulate an agent that could improve soil conditions. The above examples are not meant to be an exhaustive list of potential uses for the capsule of the innovation. It is to be appreciated that the capsule may be used in many environments wherein a change in pH (e.g., a change to low or high pH) is indicative of a need to administer/release an agent.

In one embodiment, the pH-sensitive release system comprises a corrosion inhibitor for corrosion protection. The corrosion inhibitor may be loaded into a capsule that can respond to both low and high pH conditions. In one embodiment, the capsule may be a nano-/micro-capsule. A change in pH may be indicative of conditions that can lead to corrosion. This change in pH results in release of the encapsulated corrosion inhibitors. This is in contrast to agents directly embedded inside a barrier coating as the corrosion inhibitor loaded into a capsule is controllably released depending on pH to minimize the inhibitor depletion.

In one embodiment, the pH-sensitive release system according to the innovation may include an agent embedded into a barrier polymer matrix to achieve a smart coating for corrosion protection. There are a variety of applications for such a smart coating, including automotive, aerospace, marine and biomedical components. In one embodiment, the agent may be a corrosion inhibitor. By choosing appropriate corrosion inhibitors, this coating can be used for protection of a variety of metal substrates (e.g., Al, Mg and Cu and their alloys). The pH-sensitive release system according to the innovation may include additional functionality. For example, if used in a coating, the system could provide early detection of corrosion by impregnating a pH indicators inside a capsule. It is to be understood that the pH-sensitive release system according to the innovation may be used to detect changes in pH in most any suitable environment.

The capsules (e.g., PEI/PAA capsules) according to the innovation can release agents (e.g., corrosion inhibitors) much faster at low or at high pH compared to neutral pH. In environment prone to changes in pH, especially those prone to corrosion, these capsules are suitable for providing corrosion protection for substrates as a whole, especially metal substrates.

In one aspect, the innovation provides a corrosion inhibitor release system comprising an encapsulated corrosion inhibitor. In one embodiment, the corrosion inhibitor may be encapsulated within a micro-container or nano-container. In one embodiment, the micro-container or nano-container may be built using at least two weak polyelectrolytes. The weak polyelectrolytes may include a weak polycation and a weak polyanion. In one embodiment, the polyelectrolytes may be polyethylenimine (PEI) and polyacrylic acid (PAA).

In an aspect, the innovation provides a method of fabricating a capsule for encapsulating an agent. In one embodiment, the agent may include a corrosion inhibitor. In one embodiment, the method includes mixing two weak polyelectrolytes. In one embodiment, the method includes mixing a weak polycation and a weak polyanion (e.g., polyethylenimine (PEI) and polyacrylic acid (PAA)) to build a micro-container or nano-container for corrosion inhibitors. Upon mixing, the polyelectrolytes (e.g., PEI and PAA) can interact with each other via electrostatic interaction to form coacervates. The coacervates can stably exist and a homogenous solution can be made without phase separation.

In one embodiment, PEI and PAA are used to fabricate nano-/micro-capsules for encapsulation of corrosion inhibitors. PET and PAA are weak polyelectrolytes and carry positive (PEI) and negative (PAA) charge. Upon mixing, PEI (e.g., 80 mM with respect to amine groups) and PAA (e.g., 80 mM with respect to carboxylic acid groups) can interact spontaneously with each other via electrostatic interaction to form coacervates. Coacervates can stably exist and a homogenous solution can be made without phase separation by modifying the pH of the solutions and the ion concentration. In one embodiment, the pH of the solutions is controlled by adding acetic acid to have stable coacervates when PEI and PAA are mixed together.

The degree of ionization of PEI and PAA is pH sensitive. The pKa values of PEI are 4.5, 6.7 and 11.6 while the pKa of PAA is 5.5. At low pH (e.g., less than about 5.5) PEI carries more positive charge and PAA becomes neutral due to protonation. At high pH (e.g., greater than about 11), on the other hand, PEI loses charge and PAA is fully ionized and becomes more negative. In both low and high pH environments, the interaction between PEI and PAA becomes weaker and repulsion between species with the same charge becomes stronger, inducing the swelling or dissolution of PEI/PAA coacervates. As a result, inhibitors enclosed within a capsule comprising PEI/PAA coacervates can be released.

In one embodiment, the pH response of PEI/PAA coacervates can be modified by adjusting the molar ratio of PEI and PAA. As described in the Example below, three molar ratios were tested to determine pH response of the PEI/PAA coacervates. In one embodiment, the molar ratio of PEI/PAA may be selected from about 2:1, about 1:1, or about 1:2.

In one example, a release system according to the innovation was fabricated and tested. It was observed that the release of an organic dye from a film made by PEI/PAA coacervates was much faster at either low (2.5) or high pH (11) compared with neutral (7) pH.

According to an aspect, the innovation provides an electrospray method for fabricating a capsule that is pH-responsive. In one embodiment, PEI/PAA coacervates and corrosion inhibitors can be loaded into outer and inner tubes of an electrosprayer, respectively and, thus, micro- or nano-containers with a core-shell structure can be fabricated. In contrast with the traditional technique for fabrication of nano-containers (e.g., the layer-by-layer technique), the method according to the innovation is fast and easy and able to directly encapsulate any functional species. In one embodiment water-soluble salts may be encapsulated inside a polymeric capsule with high loading efficiency using a method according to the innovation.

To fabricate nano-/micro-capsules, there are numerous techniques including the layer-by-layer technique and in situ polymerization. These techniques, however, are time-consuming and suffer from a narrow range of feasible materials as well as low loading efficiency. When it comes to capsules made using polyelectrolytes, the layer-by-layer technique is most often used.

In one embodiment, a method according to the innovation includes the preparation of prepared polyelectrolyte coacervates to make capsules using electrospray techniques to fabricate core-shell structured capsules. This method is more cost- and time-efficient than currently sued methods. Compared with existing techniques used for fabricating capsules (e.g., the layer-by-layer technique), preparation of polyelectrolyte complexes can be quickly finished by mixing two polyelectrolytes together, which, in some cases takes only seconds. Thus, the tedious and time-consuming preparation of polyelectrolyte multilayers can be avoided using methods according to an aspect of the innovation.

In one embodiment, for example, using the electrospray technique to enclose inhibitors within polyelectrolyte capsules can form core-shell structured capsules once solutions are ejected from an electrospray nozzle. In one embodiment, the electrospray technique may utilize an electrospray apparatus having multiple nozzles, thus allowing for capsule creation through multiple nozzles at the same time.

In one embodiment, the electrospray method is used to encapsulate corrosion inhibitors within PEI/PAA coacervates. In one embodiment PEI/PAA coacervates (0.5 wt %) in dichloromethane (DCM)/ethanol were prepared and sodium vanadate (NaVO₃) (0.1 M) in DI water was used as the corrosion inhibitor. DCM is an organic solvent used in electrospray and ethanol can help with fabricating stable, liquid-like PEPPAA coacervates in organic solvents. In one embodiment, a coaxial electrospray nozzle may he used to fabricate core-shell structured nano-/micro-capsules.

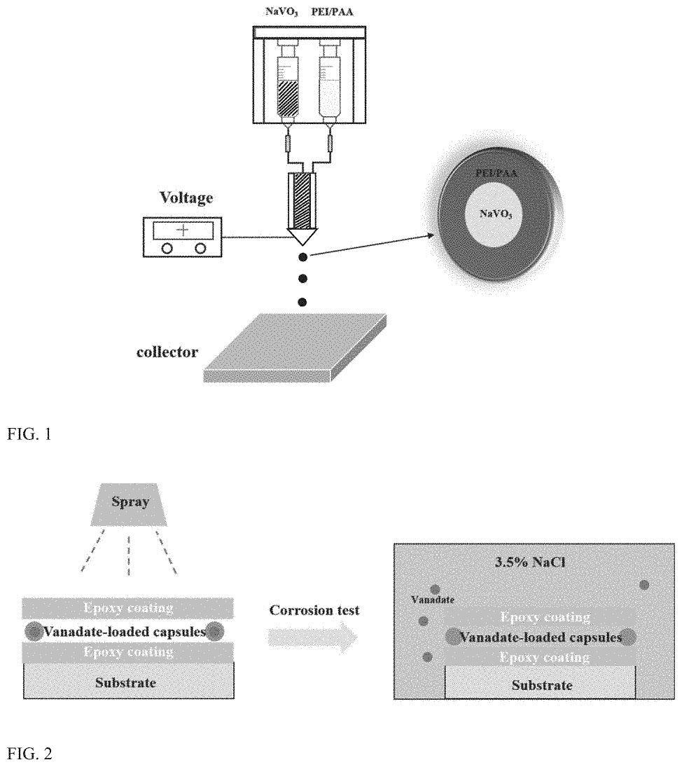

As shown in FIG. 1, in one embodiment, PEI/PAA coacervates are filled in the outer tube and NaVO₃ solution is in the inner tube of an electrosprayer so that PEI/PAA coacervates can form a polymer shell that is impregnated with NaVO₃. In the embodiment depicted in FIG. 1, the distance between the nozzle and the collector is set at 15 cm. The size of the resulting capsule and the thickness of the polymer shell may be modified by controlling the voltage applied to the coaxial nozzle and the outer and inner flow rates. It is to be appreciated that the NaVO₃ solution is but one example of an agent that may be encapsulated by the PEI/PAA coacervates. As described herein, the encapsulated agent is selectable.

In one embodiment, the nano-/micro-capsules may be combined with most any commercially available coating to protect a substrate. in one embodiment, the nano-/micro-capsules may be combined with a coating to provide corrosion protection for various substrates (e.g., metals, ceramics, etc.). To achieve corrosion protection of a metal substrate, these nano-/micro-capsules can be combined with any commercially available coating, such as epoxy and polyurethane coatings.

In one embodiment, the method may include electrospray technique to fabricate a. corrosion protection system with a sandwich structure as depicted in FIG. 2. In this embodiment, an organic coating, e.g. an epoxy coating, may be sprayed onto a metal substrate (e.g., aluminum). Nano-/micro-capsules encapsulating a corrosion inhibitor may then be electrosprayed on top of the coating. In one embodiment, the nano-/micro-capsules may be vanadate-loaded nano-/micro-capsules. Another layer of coating (e.g., an epoxy coating) by spraying may be deposited on top of the nano-microcapsule layer.

In one embodiment according to the innovation, a wide variety of corrosion inhibitors can be impregnated/encapsulated within PEUPAA coacervates. This can be accomplished while minimizing limitations associated with choosing proper inhibitors and solvents found with prior techiques. There are significant limitations associated with choosing inhibitors for existing inhibitor-loaded capsules preparation methods. For example, insoluble inhibitors or inhibitor-loaded templates are required if the layer-by-layer technique is used. Water-soluble inhibitors are required if water-in-oil emulsion is used to fabricate inhibitor-loaded capsules. In contrast, the electrospray technique according to the innovation uses a coaxial nozzle so that inhibitors and materials used for fabricating capsule shells separately flow through the inner tube and outer tube of the coaxial nozzle, respectively, minimizing the interaction between inhibitors and shell materials. Hence, a wider range of inhibitors can be used. In one embodiment, the corrosion inhibitor is sodium vanadate (NaVO₃).

Use of the electrospray technique according to the innovation can also address the issue of loading efficiency. The amount of inhibitors encapsulated within capsules using prior methods (e.g., layer-by-layer) has been found to be low in most studies (20-30%), while methods utilizing the electrospray techniques according to aspects of the innovation may enhance the loading efficiency to over 50%.

In one embodiment, the size of PEI/PAA capsules is controllable and PEI/PAA capsules are self-sealable. Defects or voids formed by the consumption of encapsulated inhibitors during the release process can create a potential pathway for electrolytes in a corrosive environment to penetrate the coating and interact with the metal substrate, causing local corrosion. To address this issue, in one embodiment, pore size may be controlled within a certain range to render a desired corrosion protection performance. Compared with other techniques, the size of capsules fabricated by electrospray is easier to be adjusted by controlling voltage and flow rates. In addition, previous studies have used strong polyelectrolytes as components of capsules while PEI and PAA used according to the innovation are weak polyelectrolytes. In addition, these weak polyelectrolytes have higher mobility when they are wet. Thus, in one embodiment, the PEI and PAA may diffuse with each other and seal voids/defects generated by the depletion of inhibitors.

### EXAMPLE

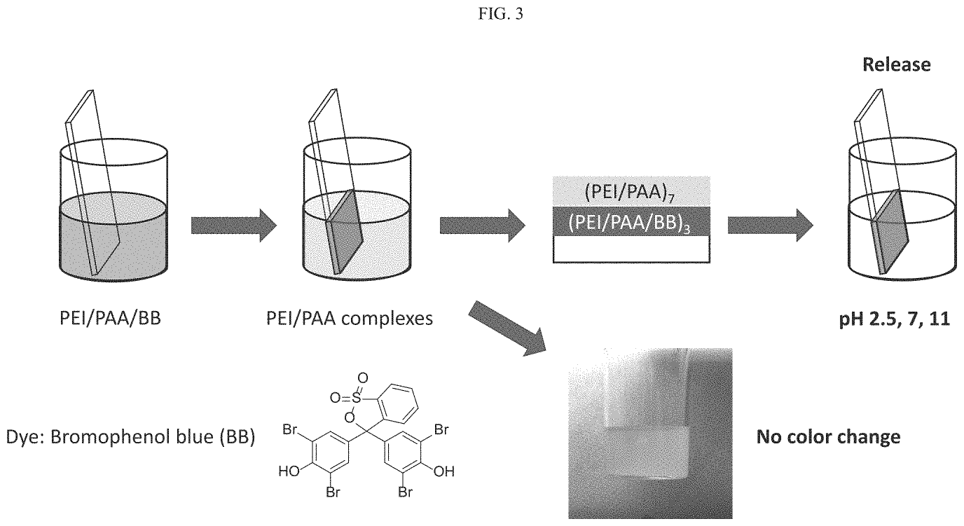

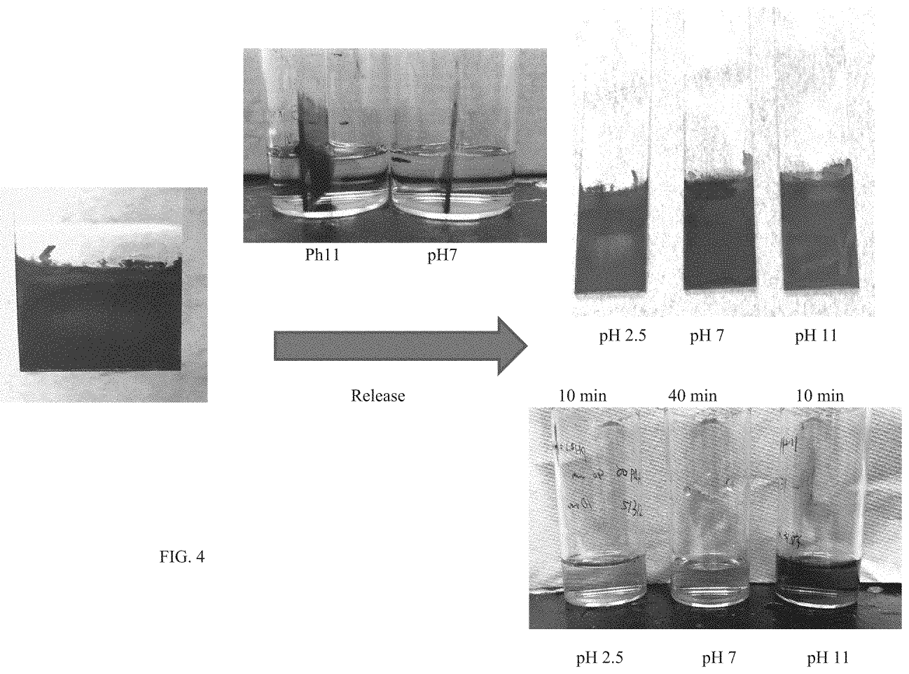

PEI/PAA complexes having three different molar ratios were tested to determine timing of release in different pH environments. PEI/PAA complexes having molar ratios of 1:1, 2:1, and 1:2 were tested. (FIG. 3.)

Release of an organic dye (brornophenol blue) from glass slides coated with PEI/PAA complexes was faster in low pH (2.5) and high pH (11) as compared with a neutral pH (7) for all of the tests. (See FIGS, 4-6.)

Release of the dye at pH 7 was very slow using PEI/PAA having a molar ratio of 1:1. As seen in FIG. 4, there was no color change in the solution after releasing for 40 minutes. Release at pH 2.5 and 11 was much faster. (See FIG. 4.)

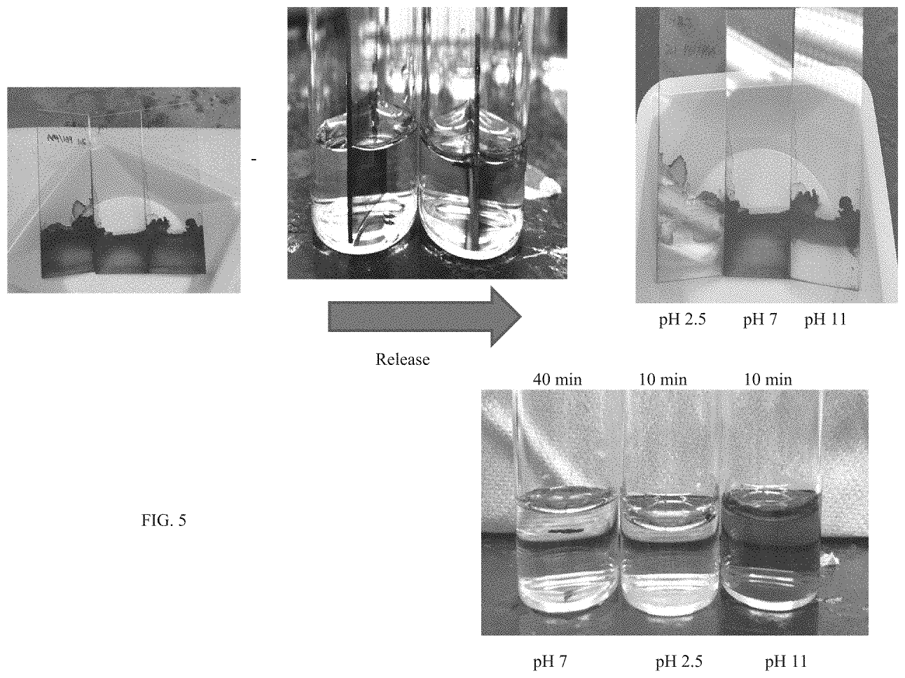

PEI/PAA with a molar ratio of 2:1 also showed that the release rate at pH 2.5 and pH 11 was faster than release at pH 7. Release at pH 11 was faster than release at pH 2.5. (FIG. 5).

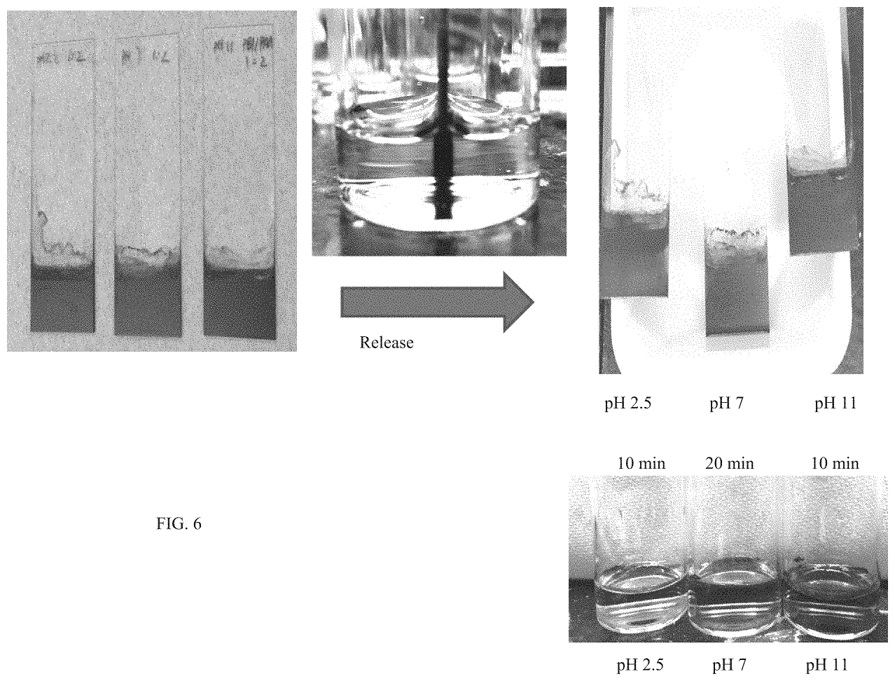

PEI/PAA with a molar ratio of 1:2 also showed that the release rate of the dye at pH 2.5 and pH 11 was faster than at pH 7. The release at pH was faster and a diffusion layer was observed after releasing for 20 minutes. (FIGS. 6.)

What is claimed is:

1. A pH-sensitive release system comprising:
a capsule comprising at least two weak polyelectrolytes that responds to both low pH and high pH changes and
an agent encapsulated with the capsule,
wherein the agent is released from the capsule when an environmental pH level changes to either a low pH or a high pH.

2. The pH-sensitive release system of claim 1, wherein at least two of the polyelectrolytes comprise polyethylenimine (PEI) and polyacrylic acid (PAA).

3. The pH-sensitive release system of claim 2 wherein the agent is a corrosion inhibitor.

4. The pH-sensitive release system of claim 3 wherein the capsule is a nano-capsule or a micro-capsule.

5. The pH-sensitive release system of claim 2 wherein the molar ratio of PEI to PAA is about 1:1.

6. The pH-sensitive release system of claim 2 wherein the molar ratio of PEI to PAA is about 2:1.

7. The pH-sensitive release system of claim 2. wherein the molar ratio of PEI to PAA is about 1:2.

8. The pH-sensitive release system of claim 1 wherein the system further includes a coating comprising the capsule.

9. The pH-sensitive release system of claim 8 wherein the coating is a coating for a metal substrate.

10. A method of forming a pH-sensitive release system comprising:
preparing a coacervate comprising at least two weak polyelectrolytes;
filling one portion of an electrospray apparatus with the polyelectrolyte complex;
filling a second, separate portion of the electrospray apparatus with an agent; and
ejecting the polyelectrolyte complex and the agent through a coaxial nozzle operatively connected to the electrospray apparatus,
wherein the coacervate forms a capsule having a polymer shell that is impregnated with the agent.

11. The method of claim 10, wherein at least two of the weak polyelectrolytes comprise polyethylenimine (PEI) and polyacrylic acid (PAA).

12. The method of claim 11, wherein the agent is a corrosion inhibitor.

13. The method of claim 12 wherein the agent comprises sodium vanadate.

14. The method of claim 10 wherein the pH-sensitive release system comprises:
a first layer of an organic coating;
the capsule; and
a second layer of the organic coating,
wherein the capsule is between the first layer and the second layer.

15. The method of claim 14 wherein the organic coating is an epoxy coating.
